(19) United States
(12) Patent Application Publication
(10) Pub. No.: US 2022/0110572 A1
(43) Pub. Date: Apr. 14, 2022

(54) Magnetic Brain Computer interface Surface Membrane

(71) Applicant: Austin James Georgiades, Marlborough, CT (US)

(72) Inventor: Austin James Georgiades, Marlborough, CT (US)

(21) Appl. No.: 17/069,866

(22) Filed: Oct. 14, 2020

(51) Int. Cl. A61B 5/291; A61B 5/369; A61B 5/271

(52) U.S. Cl. CPC A61B 5/291; A61B 5/271; A61B 5/369

(57) ABSTRACT

A flexible, artificial membrane circuit comprised of controlled electromagnetic coils placed in contact with the surface of tissue-capable-of-generating-an-action-potential for measurement and stimulation of activity in discrete regions of tissue. Circuit includes control systems, power systems and wireless communication systems as well to allow coil stimulation activity to be controlled by an outside source and to relay measurements of activity to an outside device.

The patent being described here is a flexible, artificial, circuit-membrane comprised of layered, electromagnetic (EM) coil arrays designed to stimulate and measure the activity of different regions of tissue-capable-of-generating-an-action-potential (APT) and the necessary control and communications circuitry needed to make it a brain computer interface. APT can be defined as any number of cells greater than or equal to one which are capable of generating an action potential in response to an applied EM field. An action potential herein is defined as an electrical current across a cell membrane which causes current to pass across other regions of cell membrane. Membrane, as defined herein, is a broad, relatively thin, surface or layer.

In principle, this patent uses the same underlying principles as transcranial magnetic stimulation (TMS). In TMS, EM coils (also referred to as inductors) of conductive wire are arranged to stimulate regions of brain tissue from outside the skull using a dynamic magnetic field to induce a current in the target region of brain tissue and thus induce an action potential. The surface position, magnetic field strength, coil geometries and relative angles between the two coils determine the three-dimensional flux distribution of the applied magnetic field. The region within the magnetic field with the highest flux density shall be referred to as the focal region, and in the case that the magnetic field is dynamic, this region experiences the greatest magnitude of induction for a magnetic field of any strength. For any non-zero magnetic field, if the magnitude of oscillation in the field is increased from zero, it is this focal region of highest flux density that will first reach the threshold of depolarization for an APT. By calibrating the strength of induction to cause only a discrete group of cells within this focal region to depolarize, a pair of electromagnetic coils can be used to target a specific region of APT for stimulation.

EM coils, when arranged in series with a current monitor, can also be used to measure changes in EM potential. The ability for an EM coil to measure changes in EM potential depends on both the magnitude of the change in EM potential (induction) as well as the position of the source of said change relative to the detection coil(s). As a rule, the relative strength of induction that a current has on a measuring coil is proportional to the distance the current oscillation is away from the measuring coil. This means that current oscillations positioned closer to the coil passively recording inductance will induce a greater magnitude of current in the measuring coil than current oscillations of the same magnitude positioned further away. In order to locate EM potential changes of unknown magnitude within a discrete region of three-dimensional substrate, a total of 4 coils (or three coil pairs, each sharing one of the same coils) must be used which vary in their x, y and z coordinates. In what is essentially the reverse process of GPS trilateration, the relative strength of a signal as compared across the readings 4 coils can be used to position the source within three dimensions. Thus, a three-dimensional matrix of EM coils can be used to measure the electrical activity of APT.

By arranging EM coils in arrays across the surface of APT and controlling the current that flows through them, the number of regions of tissue that can be stimulated increases. By arranging multiple layers of EM coil-arrays across the surface of APT, the volume of tissue that can be accurately measured increases (along with the potential precision of the measurements). The systems necessary to individually control EM coil activity, including controlling current strength and direction, as well as controlling the switch between active-stimulation-mode and passive-measurement-mode would be incorporated into circuitry. Coordinated, higher control over these individual EM coil functions would enable stimulation of many discrete regions of APT and allow accurate measurement of activity across a wide volume of APT.

For example, to further increase the number of discrete, point-like regions that can be stimulated, EM coil arrays can be stacked on top of each other to form a three-dimensional, laminar structure called a coil matrix. EM coil size, geometry, spacing between coil centers and angle relative to surface may all be modified, if necessary, to produce the desired distribution of focal points within the target tissue. For example, EM coils with greater spacing between their centers have focal points further away from the plane of the coils. Thus, EM coils with greater spacing between their centers would be used to stimulate and measure deeper regions of tissue. Coils located within the same layer of membrane can stimulate an array of focal points at a certain depth when paired only with adjacent coils, but can also stimulate deeper regions of APT as well by pairing with coils further away. Although the magnitude of the current change in these non-adjacent coils would need to be larger than when adjacent to stimulate the pair's focal region, being able to vary the magnitude of current supplied to coils enables arrays of focal points at different depths to be stimulated from a single layer of EM coils.

In theory, the number of distinct focal regions that can be stimulated by an array of EM coils is equal to the number of unique combinations of two coils that can be formed. In practice, the inability to provide enough current for coils to stimulate their respective focal region when separated by a large distance prevents this from being made manifest. For an given coil, there is a certain radius where combinations of coils (which include said coil) are capable of stimulating their respective focal region. Working to increase the magnitude of current that can be supplied to coils extends the maximum distance between coils in which stimulation of the focal region is still possible. In addition, the focal region stimulated can be enlarged by increasing the current flowing through the EM coil-pair which have that focal region, thereby increasing the volume of tissue where induction is above the threshold needed to induce an action potential, thus the volume of the region stimulated can be modulated. The range of volume that can be stimulated by a coil-pair depends on the range of current which can be supplied to coils, thus coil-pairs which are far apart have less dynamic range in the volume of the region stimulated than coils which are close together.

In theory, EM coil arrays do not need to be static nor laminar. For example, it is possible to have a dynamic, laminar model comprised of a grid of squares with 4-way junctions at each of the vertices. Control systems would require a means of controlling how current flows through vertex junctions in addition to controlling current strength and being able to switch between measurement and stimulation. The ability for dynamic junctions to control the circuit of the EM coil means that a grid of square EM coils can produce more possible circuits than the elemental square units that comprise it. Going even further, at the theoretical extreme, a cubic matrix with 6-way junctions at the vertices could even further increase the number of possible circuits (and therefore magnetic fields and therefore focal regions) which could be generated from a given membrane. A membrane such as this would constitute a non-laminar, dynamic model.

The Magnetic Brain Computer Interface Surface Membrane (MBCISM), is comprised of a flexible, circuit-matrix containing EM coils on the side facing the APT and the necessary control systems required to stimulate and measure focal regions of APT, including circuitry capable of sending signals to and receiving signals from outside devices, where space allows. In addition, it would include the necessary circuitry to power the device, with power coming by external, inductive charging or by an internal power supply mechanism (see: Biological Battery, Application: 17069867).

While the MBCISM would work on all APT, it is optimized for use as a brain-computer interface. Its design allows for easy implantation and removal (see: Magnetic Brain Computer Interface Surface Membrane Injector, Application: 63069307) and unlike electrodes, there is no need to avoid blood vessels and nothing is physically disturbing the neural tissue matrix. Perhaps the greatest benefit, however, is the ability for membranes to cover a large surface area of brain and read/write to a large number of focal points within neural tissue. Constant output of brain-state information from measurement across the volume of the cortex can be used to gather a large amount of detailed data from across a very wide region of the brain. This data can then be analyzed computationally and used to generate input that can modulate brain activity. The input space is maximized by the large number of focal points within the brain tissue that can be stimulated. Thus, the MBCISM is optimized for use as a brain computer interface.

### DRAWING

FIG. 1:

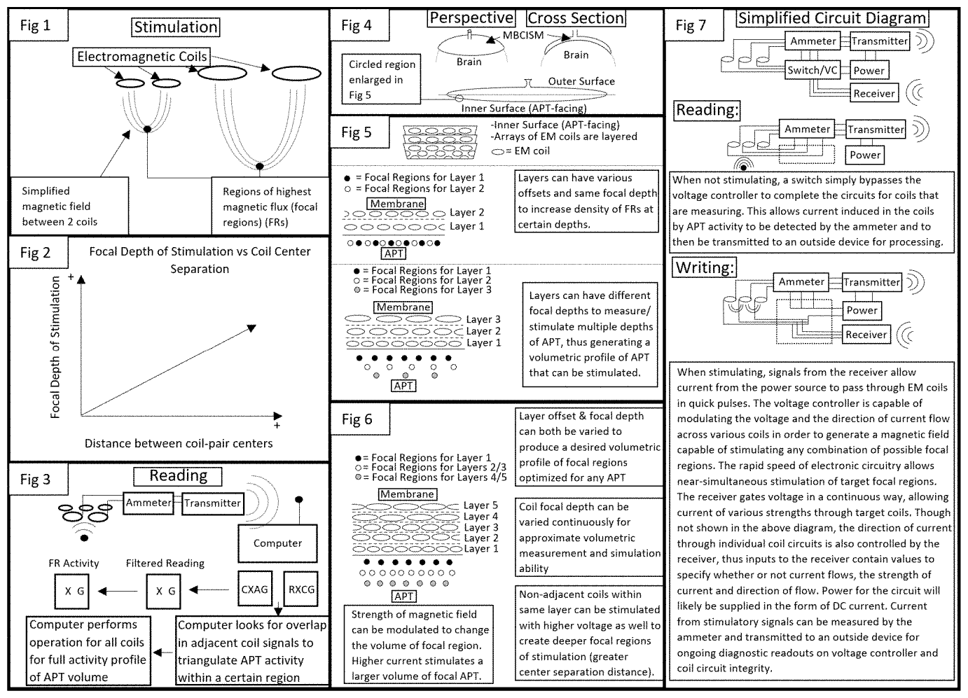

The illustration in FIG. 1 shows two pairs of electromagnetic coils and a portion of their respective magnetic fields in the case that the coil-pair receive current that passes in opposite directions to each other (so as to produce magnetic dipoles which are opposite in direction). The dots represent the regions of highest magnetic flux within the illustrated magnetic field. If one were to increase the magnitude of change in the magnetic field strength from zero, the first region of APT to reach stimulatory threshold is within the region of highest magnetic flux, which is termed the “focal region” (FR). This demonstrates the principle by which a discrete region of APT can be stimulated by a dynamic magnetic field between two EM coils.

FIG. 2:

The illustration in FIG. 2 illustrates the relationship between the separation between coil-pair centers and the distance of the focal region for said coil pair from the plane of the coils. This distance is termed “focal depth”. There is an increase in the depth of the focal region as coil-pair center separation increases. This shows how various depths of APT can be stimulated by EM coils of varying distances between their centers.

FIG. 3:

FIG. 3 illustrates the process by which the activity of APT can be determined using computational analysis of output signals. First, activity within APT (in this case, at a focal region) induces current in EM coils. This is detected by the ammeter and the current values are transmitted to an outside computer system for analysis. For simplicity, the illustration in FIG. 3 shows this process within two dimensions using only two coils, with the source located at equal distance from both coils shown. This computer system compares the ammeter readings of adjacent coils to look for signal overlap and differences in signal amplitude. In two dimensions, the sources of signals which appear roughly equal in amplitude between the readings of two adjacent coils can be located along a line, perpendicular to the line between the two coil centers, which intersects the midpoint of the line connecting both coil centers. In three dimensions, they exist on a plane, perpendicular to the line between the two coils, which passes through its midpoint.

Examination of the relative strengths of signals across four or more coils (assuming three or more coils do not share the same coordinate value in any dimension) can be used to calculate source location within a three-dimensional volume of APT. This is done by examining the relative amplitudes of discrete signals across the four coils. Usually, the coil-readings chosen by the computer to best calculate the location of region of activity are the readings of the three coils that frame the area directly over the region of activity, and a coil in the plane above them. In this example, signals are represented as strings of letters, with the same letters indicating the same signal being detected by both coils and their position left to right representing their occurrence within time. Non-similarities represent signals picked up by one coil at a certain time but not the other. In this way, discrete signals detected by two coils can be filtered to provide a profile of the activity occurring within the region between the two coils.

FIG. 4:

The perspective illustration shows what an MBCISM would look like when in operational contact with an exposed brain, resting much like a deflated whoopee cushion adjacent to the APT it interfaces with. The cross-sectional illustration demonstrates a hollow interior with a face in contact with the APT surface (proximal) and a face which is directed away (distal). Control, communication and power systems would be located on the distal surface while EM coil arrays would be on the proximal surface. The small, hollow protuberance on the top of the MCBISM is necessary for the implantation and sub-cranial inflation of the device within subarachnoid space. It is shown as being open and vertical in this illustration. It is made of the same flexible material as the rest of the circuit and serves as a means by which fluid can be pumped into the MBCISM to inflate it when under the skull. After filling, it would be pinched off, permanently sealed and laid flush with the rest of the membrane. One of the key advantages of the MBCISM is that the flexible membrane can be inserted through a relatively small hole in the skull and then inflated to cover a much wider area, thus simplifying the implantation operation vs electrodes, reducing risk, scarring and recovery time. The enclosed volume of fluid within the membrane would be under enough pressure to keep the proximal surface of the MBCISM gently but firmly pressed against the pial surface of the brain and the distal surface against the arachnoid and dura mater of the cranium, thus ensuring a good interface.

Notice, a section of the proximal surface of the MBCISM is circled and enlarged at the top of FIG. 5.

FIG. 5:

This illustration at the top of FIG. 5 shows a magnified cross-section of a portion of the proximal side of the MBCISM, showing layered arrays of EM coils that together form a composite matrix. Below are illustrated the various ways that coil-layers can be arranged to achieve certain results. The next illustration down shows multiple layers of EM coils (with offset) that share the same focal depth for increased read/write resolution at that depth. Coil layers within the circuit membrane are labeled and their respective focal regions within APT are shown. It should be noted that the solid line separating the coil layers from the focal regions demarcates the transition between MCBISM and APT and the dashed line is used to demarcate the boundary between coil-layers. The lowermost illustration in FIG. 5 shows multiple layers of EM coils trained to different focal depths for measurement and simulation across multiple focal depths of APT.

FIG. 6:

The illustration in FIG. 6 shows how coil-layers of varying offsets and focal depths can be arranged in many ways to create a custom distribution of focal regions at varying depths of APT. It also highlights the fact that focal depth can be almost continuously varied to create an ideal volumetric distribution of focal regions. This custom distribution of focal regions can be optimized for various APT to create the best possible interface for a target tissue. Although the focal regions shown for this and FIG. 5 are only those formed by pairing between adjacent coils, it should be noted that other focal regions can be stimulated too via pairing of non-adjacent coils (which includes coils within different layers). In addition, although the size of the focal regions in this illustration is shown as being the same for all coils, the size of the focal region is variable. Increasing the magnitude of change in the current passing through a coil pair (and thus increasing the strength of the current induced by the field) beyond what is required for threshold stimulation causes the region of APT being stimulated a coil pair to increase in volume, thus the size of focal regions can be varied as well.

FIG. 7:

This series of illustrations show a simplified circuit diagram of a MBCISM to illustrate how reading and writing occur. The illustration at top represents a simplified circuit diagram, showing three EM coils connected to both an ammeter and a voltage controller/switch. The ammeter is connected to a transmitter, which sends readings wirelessly to an outside device using energy from the power supply. The voltage controller/switch, meanwhile, is connected to the power supply and a receiver. The receiver gives the voltage controller/switch signals as to what EM coils to send current to, the magnitude of the current being sent and the direction of the current.

When reading, a switch simply bypasses all of the voltage control circuitry and closes the circuits for the EM coils. Circuit for all coils run through an ammeter, so current induced in the EM coils by APT activity can be measured and transmitted to an outside device. It should be noted that not all circuitry within the voltage controller (dashed box below the ammeter) is shown here. Instead, only relevant circuits are illustrated for the sake of simplicity. The function of MBCISM circuitry with regards to reading is to collect the greatest number of coil-readings possible from across as wide a volume of APT as possible, with the highest possible resolution for induced-current measurements.

When writing, the receiver sends signals to the switch to connect certain EM coils to the voltage controller. For simplicity, the control of current from the power source to various coil circuits is illustrated in the diagram by the points of contact between the output from the receiver and the power supply to the coils. However, the receiver not only controls which coils have current running through them, but also how much current in what direction. Circuitry responsible for controlling current magnitude and direction is not shown explicitly in the diagram, but occurs within the voltage-controller/switch complex with input from the receiver. Gating of current occurs via transistors controlled by the receiver. The writing function of MBCISM circuitry is to operate as a system capable of inducing action potentials in as many variably-sized, discrete regions of APT as possible within a set amount of time. The determination of which coil-pairs should be stimulated, how much current should be sent through each coil pair, and the direction of current that passes through each coil is made by an outside computational device.

1. A flexible, biocompatible, artificial membrane comprised of:
a. A flexible circuit comprised of:
i. Flexible, electronic circuitry comprising an array or arrays and/or a lattice or lattices of electromagnetic coils.
ii. Flexible, electronic circuitry comprising control systems for said electromagnetic coils.
1. Systems which control whether each coil is stimulating and/or measuring.
2. Systems which control the current flowing to each coil when stimulating.
3. Systems which link each coil in series with a current monitor when measuring.
4. Systems which control current flow at vertices in the case of dynamic coil arrays or lattices.
iii. Flexible, electronic circuitry comprising a wireless communication system or systems capable of sending information to and receiving information from an outside device.
iv. Flexible, electronic circuitry capable of controlling power supply to power the overall circuit
b. A flexible circuit matrix
c. A biocompatible outer layer

2. A system of flexible, electronic circuitry capable of measurement and stimulation of electrical activity in discrete regions of tissue-capable-of-generating-an-action-potential (APT) using electromagnetic coils within a biocompatible, artificial membrane in contact with the APT.
a. A process by which electromagnetic coils, within a biocompatible, artificial membrane in contact with APT, are used to stimulate APT at multiple, discrete locations across an area and/or at varying tissue depths.
b. A process by which electromagnetic coils, within a biocompatible, artificial membrane in contact with tissue, are used to measure the electrical activity of APT at multiple, discrete locations across an area and/or at varying tissue depths.
c. A process by which multiple arrays and/or lattices of electromagnetic coils are layered to increase the number of discrete locations within APT that can be stimulated and measured.

3. A system where the geometries of electromagnetic coils, within a biocompatible, artificial membrane in contact with tissue, are dynamically controlled:
a. A process by which the passage of current through the vertices of a two-dimensional array or arrays and/or a three-dimensional lattice or lattices of electromagnetic coils is dynamically controlled.
i. A process by which current entering a vertex is allowed or disallowed to pass through each of the possible exit paths.

4. A process by which a wireless communications system receives and transmits information from and to an outside device.

5. A process by which a coil control system, outlined in claim 1. a. ii., passes current through various coils to stimulate desired foci in response to input instructions.

6. A process by which a coil control system, outlined in claim 1. a. ii., outputs induced current readings from various coils to measure the electromagnetic activity of desired foci in response to input instructions.

7. A process by which a coil control system, outlined in claim 1. a. ii., controls the magnitude of current applied to electromagnetic coils.

8. A power system which provides the necessary energy to power the circuit.
